(19) United States
(12) Patent Application Publication
(10) Pub. No.: US 2017/0247652 A1
(43) Pub. Date: Aug. 31, 2017

(54) Device and Method for High Throughput Bacterial Isolation

(71) Applicant: Northeastern University, Boston, MA (US)

(72) Inventors: Edgar D. GOLUCH, Somerville, MA (US); Nil TANDOGAN, Cambridge, MA (US)

(21) Appl. No.: 15/516,911

(22) PCT Filed: Oct. 20, 2015
(86) PCT No.: PCT/US2015/056412

§ 371 Date: Apr. 5, 2017

Related U.S. Application Data
(60) Provisional application No. 62/065,944, filed on Oct. 20, 2014.

(51) Int. Cl. C12M 1/26; C12N 1/20; C12M 1/34; C12M 3/06; C12M 1/12; C12N 1/02; C12M 1/32

(52) U.S. Cl. CPC C12M 33/14; C12N 1/02; C12N 1/20; C12M 23/12; C12M 23/16; C12M 25/04; C12M 41/44

(57) ABSTRACT

Devices and methods for isolating and characterizing microbial cells from an environment are provided. The devices integrate sub-micron constrictions in a nanofluidic device with a standard microtiter plate format to facilitate the high throughput isolation, culturing, analysis, and screening of bacteria and other microbial cells in natural and man-made environments, particularly environments containing microbes adhered on particulate matter.

### STATEMENT REGARDING FEDERALLY SPONSORED RESEARCH OR DEVELOPMENT

The invention was developed with financial support from Grant No. 1353853 from the National Science Foundation. The U.S. Government has certain rights in the invention.

### BACKGROUND

Existing methods for cultivation of microbial cells from natural environments are limited. Typically, the target environment is sampled, and an inoculum from the cells contained in the sample is placed on a nutrient medium. In this process cells are removed from their natural environment to an artificial environment and manipulated prior to their exposure to a growth permissive condition. Such handling and manipulation is likely to damage cells targeted for cultivation. It is well-known that only a small fraction of cells in a sample will grow upon inoculation. Thus, there is a need to develop sampling devices that introduce a minimum of handling during harvesting of microbes from an environment. One such sampling device is the “trap” method of Gavrish, E., A. Bollmann, S. Epstein, and K. Lewis (J Microbiol Methods 72:257-262 (2008)). In that method, the growth chamber is separated from the environment by porous membranes, which contain multiple pores and lead to mixed cultures. A microbial sampling device that allows monocultures to be grown from single cells is described in WO 2013/148745A1 (which is hereby incorporated by reference). However, the device designs described there do not easily lend themselves to a high throughput screening format, or to the rapid isolation and analysis of microbes from natural environments or non-liquid environments, where direct access to the environment is required with ability to collect several thousand samples and process each one of them separately and quickly. Thus, there remains a need for devices and methods that permit the generation and analysis of large libraries of new microbial species.

### SUMMARY OF THE INVENTION

The invention provides devices and methods for integrating sub-micron constrictions in a nanofluidic/microfluidic device with a standard microtiter plate format to facilitate the high throughput isolation, culturing, analysis, and screening of bacteria and other microbial cells.

In a preferred embodiment, the device format is compatible with automated liquid handling equipment and can be used with microtiter plates having any standard number of wells (e.g., 24, 96, 384, 1536), by changing the density of design elements of the device. The devices and methods permit the rapid isolation of pure cultures of bacteria and other microbial cells from environments containing cells adsorbed onto solid surfaces, such as soil grains, sand grains, ice crystals, mineral crystals and particulate biomaterials found in air, water, or soil samples, or in decomposing biomass, such as in sewage, fecal matter, industrial waste, or agricultural waste, or found in a body of water. Particulate matter such as soil grains, ice or mineral crystals, and particulate biomass can have a diameter of about 50 μm or greater, such as at least about 100, 150, 200, 250, 300, 500, or 1000 μm. The devices and methods also enable the analysis of the sensitivity of such microbial cells to chemical agents, including antibiotics, and the selective isolation of microbial cells capable of metabolizing selected chemical substances.

One aspect of the invention is a device for isolating and culturing single cells of a population of microbial cells from an environment. The device includes a nanochannel and a food chamber within a nanofluidic/microfluidic device. The nanochannel has a first end disposed at a surface of the device, which surface is exposed to an environment that contains a mixture of microbial cells. The food chamber is fluidically coupled to a second end of the nanochannel. The nanochannel has a cross-sectional diameter that allows the entry of only a single microbial cell from the mixture of microbial cells in the environment and prevents the microbial cell from entering the food chamber, but allows progeny of the single microbial cell to enter the sterile food chamber, where they can proliferate and form a monoculture. In some embodiments of the device, the food chamber possesses an aperture at a surface exposed to the environment, and the aperture is covered with a nanoporous membrane that allows chemical substances from the environment, but not microbial cells, to enter the food chamber. Each portion of the device having a single food chamber, a single nanochannel allowing single microbial cells to proliferate into the food chamber, and optionally an aperture covered by a nanoporous membrane, defines a “microbial isolation unit”. In some embodiments, the device contains a one-dimensional array or a two-dimensional array of microbial isolation units. In a highly preferred embodiment, the device contains a two-dimensional array of microbial isolation units which are configured in a “microtiter plate format” which is compatible with commercially available robotic fluid handling devices to allow for high throughput isolation, sub-culturing, and analysis of microbial cells that grow in food chambers of the device.

Another aspect of the invention is a method of fabricating a device such as described above. The method includes the steps of: (a) fabricating a substantially planar substrate containing a nanochannel and a nanoporous aperture by the steps of: (i) providing a substantially planar silicon, glass, or quartz substrate; (ii) performing a first deep reactive ion etching from an upper side of the substrate to remove a plurality of first columns of material from said substrate, leaving a floor at a base of said columns, the floor having a thickness from about 20 to about 60 pm; (iii) performing a second deep reactive ion etching to remove a plurality of second columns of material from said substrate, each second column adjacent to one of said first columns, the second columns extending the entire thickness of the substrate, and to perforate the floor of the first columns; (iv) coating the substrate with an oxide layer, whereby the floor perforation of the first columns achieves a desired first diameter and the floor achieves a desired thickness, defining a single nanochannel in the floor of each of the plurality of first columns, each nanochannel having said first diameter and a length equal to the floor thickness, and whereby the second columns each create a plurality of apertures of a second diameter (e.g., from about 50 μm to about 500 μm, or about 100 μm to about 1000 μm, or about 500 μm to about 2000 μm), each aperture adjacent to one of said nanochannels; and (v) bonding a nanoporous membrane across each aperture at a lower surface of the substrate to form said nanoporous apertures; wherein the nanochannels and apertures form a two dimensional array corresponding to a two dimensional array of wells in a microtiter plate format; and (b) bonding the substrate from (a) to a bottom side of a microtiter plate whose wells lack floors, whereby the substrate forms floors of wells of the microtiter plate to form said device; wherein the substrate is aligned with the wells such that a single nanochannel and a single aperture are present in the floor of each well.

Yet another aspect of the invention is a method of isolating and culturing a single microbial cell to obtain a monoculture of microbial cells. The method includes the steps of: (a) depositing a device as described above into an environment containing a mixture of microbial cells such that the surface of the device containing the first end of said nanochannel contacts material of said environment suspected of comprising said microbial cells; (b) allowing one of said mixture of microbial cells to migrate into the nanochannel of the device; (c) maintaining the device under conditions suitable for allowing said microbial cell to divide within the nanochannel and produce progeny, whereby the progeny eventually enter the food chamber; and (d) maintaining the device under conditions suitable for the progeny entering the food chamber to multiply in the food chamber, forming a monoculture of microbial cells. In some embodiments the method further includes the step of (e) removing the device from said environment for analysis or sub-culturing of the microbial cells that have grown in the food chambers.

Still another aspect of the invention is a method of characterizing an effect of a chemical agent on the growth and/or survival of a population of microbial cells. The method includes the steps of: (a) forming a monoculture of microbial cells using the method described above; (b) supplying a chemical agent to the environment in which the device is deposited and allowing the agent to diffuse through the nanoporous membrane into the food chamber; and (c) characterizing an effect of the chemical agent on the physiology and/or growth of the microbial cells in the food chamber.

Another aspect of the invention is a method of characterizing an effect of a chemical agent on the growth and/or survival of a population of microbial cells. The method includes the steps of: (a) forming a monoculture of microbial cells using the method described above; (b) sub-culturing the microbial cells from (a) into a device comprising a growth chamber, the growth chamber comprising an aperture covered by a nanoporous membrane; (c) depositing the device containing the sub-culture into an environment containing or suspected of containing a chemical agent diffusible through the nanoporous membrane; and (d) characterizing an effect of the chemical agent on the physiology and/or growth of the microbial cells in the growth chamber.

Yet another aspect of the invention is a method of isolating and/or identifying a microbial species or strain that metabolizes a chemical agent or degrades a biomaterial. The method includes the steps of: (a) performing the method describe above, wherein one or more food chambers of the device are preloaded with the chemical agent or the biomaterial; (b) removing the device from the environment; and (c) analyzing, isolating, or sub-culturing microbial cells whose survival and/or growth was enhanced in the presence of the chemical agent or the biomaterial in the device.

Even another aspect of the invention is a method to aid in the identification of antibiotic-producing microbial cells. The method includes the steps of: (a) performing the method described above, wherein one or more food chambers of the device are preloaded with a target pathogenic microbe; (b) removing the device from the environment; and (c) analyzing, isolating, or sub-culturing microbial cells that overgrow the pathogenic microbe in the device.

The invention is further summarized by the following list of items.
1. A device for isolating and culturing single cells of a population of microbial cells from an environment, the device comprising:

a nanochannel comprising a first end disposed at a surface of the device exposed to an environment, the environment comprising a mixture of microbial cells; and

a food chamber fluidically coupled to a second end of the nanochannel; wherein the nanochannel has a cross-sectional diameter that allows entry of only a single microbial cell from the mixture of microbial cells and prevents the microbial cell from entering the food chamber, but allows only progeny of the single microbial cell to enter the food chamber.
2. The device of item 1, wherein the nanochannel has a cross-sectional diameter in the range from about 250 nm to about 1000 nm and a length of from about 10 μm to about 80 μm.
3. The device of item 1 or item 2, wherein the nanochannel has a cross-sectional diameter of about 700 nm.
4. The device of any of the previous items, wherein the device is configured such that said surface is capable of contacting a solid material in said environment.
5. The device of item 4, wherein the first end of the nanochannel is capable of contacting a solid material in said environment.
6. The device of item 4 or item 5, wherein the solid material is selected from the group consisting of soil, sand, biomass, sewage, sediment from a body of water, ice, and rock.
7. The device of item 6, wherein the solid material comprises particles having a diameter of about 50 μm or greater.
8. The device of item 6, wherein the solid material is a particulate solid material suspended in water or air.
9. The device of any of the previous items, wherein the food chamber comprises an aperture covered by a nanoporous membrane at said surface, the nanoporous membrane exposed to said environment in use and allowing passage of nutrients from the environment but not allowing passage of microbial cells.
10. The device of item 9, wherein the aperture has a diameter in the range from about 50 μm to about 500 μm.
11. The device of item 9, wherein the nanoporous membrane comprises pores having a diameter from about 5 nm to about 100 nm.
12. The device of item 11, wherein the nanoporous membrane is a polycarbonate or aluminum oxide membrane having pores of about 30 nm average diameter.
13. The device of any of the previous items comprising a plurality of nanochannels and a plurality of food chambers, wherein each nanochannel comprises a first end disposed at said surface and a second end fluidically coupled to a unique one of said food chambers, each coupled nanochannel and food chamber defining a microbial isolation unit.
14. The device of item 13, wherein the microbial isolation units are configured as a two-dimensional array.
15. The device of item 14, wherein the array is in a microtiter plate format.
16. The device of item 15, wherein the microtiter plate format is compatible with a robotic fluid handling device.
17. The device of item 15, wherein the array comprises 24, 96, 384, or 1536 microbial isolation units.
18. The device of item 15, wherein the food chambers are formed from the wells of a microtiter plate.
19. The device of item 18, wherein well bottoms of said microtiter plate are formed by a substrate attached to a lower surface of the microtiter plate, the substrate comprising the nanochannels.
20. The device of item 19, wherein the substrate comprises silicon, glass, or quartz.
21. The device of item 14, wherein each food chamber comprises an aperture covered by a nanoporous membrane at said surface, the nanoporous membrane exposed to said environment in use and allowing passage of nutrients from the environment but not allowing passage of microbial cells.
22. The device of item 21, wherein the apertures have a diameter in the range from about 50 μm to about 500 μm.
23. The device of any of the previous items, wherein the food chamber comprises a culture medium that supports the growth of at least one microbial cell of the population of microbial cells.
24. The device of item 14, wherein the plurality of food chambers comprise one or more culture media.
25. The device of any of the previous items, wherein the microfluidic food chamber is fluidically coupled with one or more nanofluidic and/or microfluidic channels that permit exchange of a fluid medium within the food chamber and/or harvesting of microbial cells from the food chamber.
26. The device of any of the previous items, wherein the nanochannel and food chamber are empty spaces in a solid structure comprising a polymer material.
27. The device of item 26, wherein the polymer material is polydimethylsiloxane (PDMS).
28. The device of any of the previous items, wherein the food chamber is an empty space in a polymer material, the nanochannel is an empty space in a silicon, glass, or quartz substrate, and the substrate is adhered to the polymer material such that the substrate forms a floor of the food chamber.
29. The device of any of the previous items, wherein the substrate further comprises an aperture covered by a nanoporous membrane, the nanoporous membrane exposed to said environment in use and allowing passage of nutrients from the environment but not allowing passage of microbial cells.
30. The device of any of the previous items, further comprising one or more valves, ports, holes, fluid reservoirs, pumps, vacuum lines, additional membranes, additional microfluidic channels, and/or additional nanochannels.
31. The device of item 23 that is sealed from the environment but for said nanochannel.
32. The device of item 24 that is sealed from the environment but for the plurality of nanochannels.
33. The device of item 31 or item 32 that is sterile and devoid of any viable microbial cells prior to placement in said environment.
34. A method of fabricating the device of any of the previous items, the method comprising the steps of:

(a) fabricating a substantially planar substrate comprising a nanochannel and a nanoporous aperture by the steps of:
(i) providing a substantially planar silicon, glass, or quartz substrate;
(ii) performing a first deep reactive ion etching from an upper side of the substrate to remove a plurality of first columns of material from said substrate, leaving a floor at a base of said columns, the floor having a thickness from about 20 to about 60 μm;
(iii) performing a second deep reactive ion etching to remove a plurality of second columns of material from said substrate, each second column adjacent to one of said first columns, the second columns extending the entire thickness of the substrate, and to perforate the floor of the first columns;
(iv) coating the substrate with an oxide layer, whereby the floor perforation of the first columns achieves a desired first diameter and the floor achieves a desired thickness, defining a single nanochannel in the floor of each of the plurality of first columns, each nanochannel having said first diameter and a length equal to the floor thickness, and whereby the second columns each create a plurality of apertures of a second diameter, each aperture adjacent to one of said nanochannels; and
(v) bonding a nanoporous membrane across each aperture at a lower surface of the substrate to form said nanoporous apertures;
wherein the nanochannels and apertures form a two dimensional array corresponding to a two dimensional array of wells in a microtiter plate; and

(b) bonding the substrate from (a) to a bottom side of a microtiter plate whose wells lack floors, whereby the substrate forms floors of wells of the microtiter plate to form said device; wherein the substrate is aligned with the wells such that a single nanochannel and a single aperture are present in the floor of each well.
35. The method of item 34, further comprising:

(c) filling the wells with one or more culture media; and

(d) sealing the wells to form the device of item 29.
36. The method of item 34 or item 35, wherein the bonding in step (b) comprises using an adhesive.
37. The method of any of items 34-36, wherein the bonding in step (b) comprises plasma treatment of the microtiter plate.
38. The method of any of items 34-37, wherein the bonding a nanoporous membrane across each aperture at a lower surface of the substrate in step (a)(v) comprises bonding a continuous strip of nanoporous membrane material across a plurality of said apertures arranged in a linear array.
39. The method of any of items 34-38, wherein the microtiter plate is a one-piece molded plastic article in the form of a microtiter plate but lacking well bottoms.
40. The method of any of items 34-39, wherein the microtiter plate has a format that is compatible with a robotic fluid handling device.
41. The method of any of items 34-40, wherein the microtiter plate comprises 24, 96, 384, or 1536 wells.
42. The method of item 35, wherein the wells are sealed with an optically transparent material.
43. The method of any of items 34-42, further comprising installing in the device one or more valves, ports, holes, fluid reservoirs, pumps, vacuum lines, additional membranes, additional microfluidic channels, and/or additional nanochannels.
44. A method of isolating and culturing a single microbial cell to obtain a monoculture of microbial cells, the method comprising the steps of:

(a) depositing the device of item 31, 32, or 33 into an environment comprising a mixture of microbial cells such that the surface of the device comprising the first end of said nanochannel contacts material of said environment suspected of comprising said microbial cells;

(b) allowing one of said mixture of microbial cells to migrate into the nanochannel of the device;

(c) maintaining the device under conditions suitable for allowing said microbial cell to divide within the nanochannel and produce progeny, whereby the progeny eventually enter the food chamber; and

(d) maintaining the device under conditions suitable for the progeny entering the food chamber to multiply in the food chamber, forming a monoculture of microbial cells.
45. The method of item 44, further comprising:

(e) removing the device from said environment for analysis or sub-culturing of the microbial cells.
46. The method of item 45, wherein the analysis comprises DNA sequence analysis of the microbial cells.
47. The method of item 45 or item 46, wherein the analysis comprises characterizing the metabolism or nutritional requirements of the microbial cells.
48. The method of any of items 45-47, wherein the device is maintained in the environment for a period of days, weeks, or months before removal from the environment.
49. The method of any of items 44-48, wherein the microbial cells are bacteria.
50. The method of item 49, wherein the bacteria are anaerobic bacteria.
51. The method of item 49, wherein the bacteria are Actinomycetes, Archebacteria, nitrogen-fixing bacteria, nitrifying bacteria, denitrifying bacteria
52. The method of any of items 44-51, wherein the device comprises a plurality of microfluidic food chambers, each fluidically coupled to a single channel opening on said surface.
53. The method of item 52, wherein a plurality of monocultures are obtained, each grown in a distinct food chamber and derived from a distinct single microbial cell of the environment.
54. The method of item 53, whereby monocultures of two or more different species of microbes are obtained.
55. The method of any of items 44-54, further comprising the step of:

(e) harvesting cells of the monoculture from the food chamber.
56. The method of any of items 44-55, wherein the nanoporous membrane permits entry of nutrients from said environment.
57. The method of item 56, wherein steps (c) and/or (d) are performed while supplying one or more additional nutrients through the membrane.
58. The method of item 57, wherein the microbial cells only grow and form a monoculture when the material obtained from the natural environment is placed in contact with the membrane.
59. The method of item 52, wherein the device comprises a plurality of food chambers, each food chamber containing a different culture medium, and whereby microbial cells from the mixture are identified based on their ability to grow in one or more of the food chambers.
60. The method of any of items 44-59, wherein any step of the method is monitored using light microscopy to observe the presence or identity of microbial cells in the nanochannel or the food chamber.
61. A method of characterizing an effect of a chemical agent on the growth and/or survival of a population of microbial cells, the method comprising the steps of:

(a) forming a monoculture of microbial cells using the method of item 44;

(b) supplying a chemical agent to the environment in which the device is deposited and allowing the agent to diffuse through the nanoporous membrane into the food chamber; and

(c) characterizing an effect of the chemical agent on the physiology and/or growth of the microbial cells in the food chamber.
62. The method of item 61, wherein the chemical agent is an antibiotic or is suspected of having antibiotic activity.
63. A method of characterizing an effect of a chemical agent on the growth and/or survival of a population of microbial cells, the method comprising the steps of:

(a) forming a monoculture of microbial cells using the method of item 56;

(b) sub-culturing the microbial cells from (a) into a device comprising a growth chamber, the growth chamber comprising an aperture covered by a nanoporous membrane;

(c) depositing the device containing the sub-culture into an environment comprising or suspected of comprising a chemical agent diffusible through the nanoporous membrane; and

(d) characterizing an effect of the chemical agent on the physiology and/or growth of the microbial cells in the growth chamber.
64. A method of isolating and/or identifying a microbial species or strain that metabolizes a chemical agent or degrades a biomaterial, the method comprising the steps of:

(a) performing the method of item 44, wherein one or more food chambers of the device are preloaded with the chemical agent or the biomaterial;

(b) removing the device from the environment; and

(c) analyzing, isolating, or sub-culturing microbial cells whose survival and/or growth was enhanced in the presence of the chemical agent or the biomaterial in the device.
65. A method to aid in the identification of antibiotic-producing microbial cells, the method comprising the steps of:

(a) performing the method of item 44, wherein one or more food chambers of the device are preloaded with a target pathogenic microbe;

(b) removing the device from the environment; and

(c) analyzing, isolating, or sub-culturing microbial cells that overgrow the pathogenic microbe in the device.

### BRIEF DESCRIPTION OF THE DRAWINGS

FIG. 1 shows a schematic illustration of a device for isolating and culturing microbial cells from an environment. Three microbial isolation units of the device are depicted.

FIG. 2 is a schematic illustration of a method of preparing a nanofluidic/microfluidic substrate for use in a device of the present invention. The structures are not shown to scale.

FIGS. 3A and 3B are schematic illustrations of a substrate for use in a microtiter plate embodiment of the invention. FIG. 3A shows a side view of the substrate, and FIG. 3B shows a bottom view. In this embodiment, the substrate is configured to mesh with a 24-well microtiter plate.

FIG. 4 shows a side view of a microtiter plate embodiment resulting from the bonding of a substrate such as that shown in FIGS. 3A and 3B. Three microbial isolation units are depicted. As shown in FIG. 4, the wells of the microtiter plate are open from above and ready to accept culture medium and to be sealed, which results in the device shown in FIG. 1.

FIG. 5 is a schematic illustration of a sealed device of the invention contacting a soil grain having a bacterial cell adhered to its surface. The bacterium enters the device through the nanochannel of the device.

### DETAILED DESCRIPTION OF THE INVENTION

Devices and methods of the invention combine the use of microfluidics and nanofluidics to manipulate single cells of microbes such as bacteria, algae, fungi, or protozoa, so that they can be either studied at the single cell level or cultured in controlled environments; the isolation of cells derived from more higher eukaryotes, such a mammals, or of viruses, is not performed using these devices or methods. The devices and methods of the invention are particularly suited for high throughput isolation, culturing, and analysis of microbial cells, such as bacteria, from natural environments, from degrading biomass, or from mixtures of microbial cells in artificial environments. They can be used to identify and culture new species or strains of microbes, to study their metabolism, and to identify or analyze their products, including antibiotics.

Nanofluidic devices of the invention include one or more nanofluidic channels (also referred to as nanochannels) or constrictions having cross-sectional dimension (e.g., diameter) in the nanometer range, such as from about 250 nm to about 1000 nm, or about 500 nm, 600 nm, 700 nm, 800 nm, or 900 nm, and extending in length for about 1 μm to about 50 μm or more, or from about 10 μm to about 80 μm. The constriction (region of smallest diameter) of a nanochannel for restricting entry of microbial cells can be the same over the full length of the nanochannel, or can be found in only a portion of the nanochannel, such as at the beginning, middle, or end. The nanochannels may be fluidically coupled with one or more other nanochannels or microfluidic channels (also referred to as microchannels) also present in the device, or directly with an environmental space, and the material found in such a space, present at a surface of the device. Two channels or spaces are “fluidically coupled” if a fluid in one can move freely into the other through a junction between the two, the junction allowing fluid transfer without significant leakage into other uncoupled spaces.

A key feature of the nanofluidics devices is that they also contain one or more food chambers (also referred to as “food channels”, “isolation chambers”, or “growth chambers”), each of which is fluidically coupled with one or more nanochannels. Each food chamber may optionally have a channel or opening that allows it to be supplied with a culture or growth medium for the microorganisms whose isolation is desired; the food chamber may or may not contain organic substances that serve as food for the microorganisms. The medium diffuses out through the attached nanochannel, where it can attract the microorganisms from an environment, for example, by chemotaxis. The width and length of the nanochannel are selected to serve as a constriction, allowing only one or a few single microbial cells to enter the nanochannel and/or to pass through the nanochannel.

In a preferred embodiment, the nanochannel is narrow enough so that a single microbial cell, such as a bacterial cell, can enter the nanochannel, but becomes lodged in the channel and cannot move through the channel. In that way, the cell blocks the channel from passage by other cells. Nevertheless, the cell lodged in the nanochannel is fed through the food chamber and can still divide within the nanochannel. The progeny of the lodged cell will then, usually within several hours to a day or more, make their way into the food chamber where they will establish a monoculture (i.e., a pure culture containing only microbial cells of a single species, variety, strain, or type descended from the originally lodged microbial cell but no other cells. The monoculture can then be studied within the device, or removed from the device for sub-culturing using standard microbiological techniques. The dimensions, volume, and geometry of the food chamber can be any desired size, amount, or shape as required by the user. However, the volume of the food chamber is preferably sufficient to be handled and transferred by commonly available laboratory equipment, such as in the range from about 1 μL to about 100 μL, although it can also be less, such as about 1 nL to about 1 μL, especially in the event the grown or isolated cells are intended for characterization on the device itself.

In preferred embodiments, the device is formed by bonding a substrate to an upper body. The substrate contains a nanochannel opening at a first end to a lower surface of the substrate and opening at a second end at a food chamber within or preferably above the substrate. Optionally, the substrate also contains an aperture covered by a nanoporous membrane which allows influx of chemical substances from the environment into the food chamber. In these embodiments, the upper body of the device contains a food chamber, which is in contact with the environment through the nanochannel and aperture of the substrate. One-piece embodiments are also within the scope of the invention. In such embodiments, a single material, such as silicon, quartz, glass, or a polymer is prepared with at least a nanochannel fluidically coupled with a food chamber. The devices described herein can be fabricated using any known technique, including but not limited to deep reactive ion etching, laser ablation, micromachining, injection molding, three-dimensional printing, lithography, deposition methods, and any combination thereof.

The device can have any shape or form as preferred by the user. However, the device should have one or more exposed surfaces, with one or more nanochannels opening at a surface of the device intended for exposure to an environment, each nanochannel providing access of microbial cells from an environment contacting the surface to one or more unique food chambers. A preferred form of the device is one that is suitable for use with automated fluid handling equipment. Therefore, a preferred form is a rectangular device matching the form of a microtiter plate, such as a device having a length of 127.8 mm, a width of 85.5 mm, and a height of 14.2 mm; such a device has a footprint of about 5 inches long and 3.3 inches wide, and the height can vary within the tolerance of the fluid handling equipment. In devices of such form, the lower essentially planar surface is the surface that is exposed to the environment, and the food chambers are distributed in the same manner, i.e., centered on the same locations, as the wells of a standard microtiter plate, such as one having 24, 96, 384, or 1536 wells, or another format that is compatible with automated (i.e., robotic) liquid handling equipment. Optionally, the device can be outfitted with a removable cover that seals the device from entrance by microbes and protects the entrances to the nanochannels prior to use. The user can then remove the cover just prior to placement of the device in the collection environment. The upper surface of the device also can be optionally fitted with a removable cover, in order to allow the user to fill the food chambers prior to use. Alternatively, the upper surface of the device can be permanently sealed, but provided with a thin cover or septum over each food chamber or well which can be pierced by fluid handling equipment.

The substrate can be constructed of silicon, glass, quartz, or a polymeric material; it is preferably hydrophilic so as to promote the flow of aqueous media through the fluidic channels and spaces of the device, or if hydrophobic it can be coated or plasma treated to render it hydrophilic. Silicon is a preferred material for the substrate. Voids in the substrate, such as the nanochannel and aperture, and any further optional nanochannels or microchannels, can be introduced by known techniques such as deep reactive ion etching (DRIE) or laser ablation.

The upper body of the device can be a hydrophilic polymer material. In some embodiments it is a somewhat elastic material that can be cast or spun on a master template, polymerized, and then removed by pulling it away from the template. A suitable material is polydimethylsiloxane (PDMS). In other embodiments, the upper body is formed from a rigid polymer, such as polystyrene or polycarbonate, or other polymers used in the manufacture of microtiter plates or cell culture vessels, or it can be glass, quartz, or silicon, or another material. The bottom surface of the food chambers can optionally be treated with a substance such as a protein, polysaccharide (e.g., an agar gel), or nucleic acid, or physically or chemically altered, so as to promote adhesion and/or growth of microbial cells. The body of the device is preferably transparent, or at least contains one or more transparent windows, to permit microscopic inspection of the device and monitoring of cells within the device.

Nanoporous membranes which cover the apertures and allow molecular exchange with the environment can be made of, for example, polycarbonate or aluminum oxide, or other materials, and preferably have pores of 100 nm or less (e.g., about 30 nm, or from about 5 nm to about 100 nm), that allow the diffusion of small molecules, proteins, and nucleic acids through the membrane but retain cells within the food chamber. Such membranes can be used to allow environmental chemical agents to diffuse into the food chamber to assist in the growth or maintenance of cells in the chamber, making it possible to grow and/or maintain cells that are otherwise uncultivable because their growth requirements are unknown, uncharacterized, or different from those supplied by standard or customized microbial culture media. The membranes also can be used to allow chemical agents secreted by the cells in the culture medium (antibiotics or other potentially useful substances) to be recovered for analysis or testing. Yet another use of the membranes is to allow substances to be delivered to cells present in the food chamber to test their effects on the cells, their metabolism, or their growth.

A nanochannel is fluidically coupled to the food chamber at one end of the channel and extends vertically downward, where it meets with a surface of the device and is fluidically coupled with the environment of the device near that surface, or in direct contact with that surface. The long axis of the nanochannel is preferably oriented perpendicular to the environment-exposed surface of the device, although other angles would work as well. The nanochannel opens at one end into the food chamber, preferably near the middle of one side of the food chamber, though the exact alignment is not critical. The bacteria are attracted to food slowly leaking out through the nanochannel into the environment, and they may gather in the environment at the opening of the nanochannel. This happens quickly, over minutes to a few hours after the bacteria are introduced (long before the food would entirely leak out of the food chamber, which generally would take a day or more). Because the diameter of the nanochannel is too small to allow free travel of the bacteria up into the food chamber, a single microbial cell, such as a bacterium, becomes lodged at the nanochannel opening, which prevents further bacteria from entering the nanochannel.

It is understood that nanofluidic devices of the invention can include any element or feature commonly used in microfluidic or nanofluidic devices, in microelectronic or nanoelectronic devices, or in medical devices. These include, without limitation and in any combination, one or more channels (microscale or nanoscale), reservoirs, ports, holes, valves, air-filled spaces, fluid-filled spaces, waste receptacles, pump mechanisms, vacuum lines or ports, needles, electrical devices or connections, circuitry, sensors, nanoelements (i.e., nanoparticles and/or nanotubes, assembled or free), biomolecules (including peptides, proteins, nucleic acids, sugars, antibodies, lipids, growth factors, cytokines, or metabolites), surface coatings of any kind, membranes, viewing panels, attached tubing or lines, display devices, microprocessors, memory devices, buttons, user interfaces, and wireless transmitters and/or receivers. The devices also can be adapted either for laboratory use, for field use in external natural or manmade environments, including under harsh or extreme conditions, or for implantation into the body or mounting on the surface of a human, animal, or plant, or for harvesting microbes from the air, from surfaces of buildings or inhabited spaces, from a body of water, or from a location submerged in soil, rock, or ice.

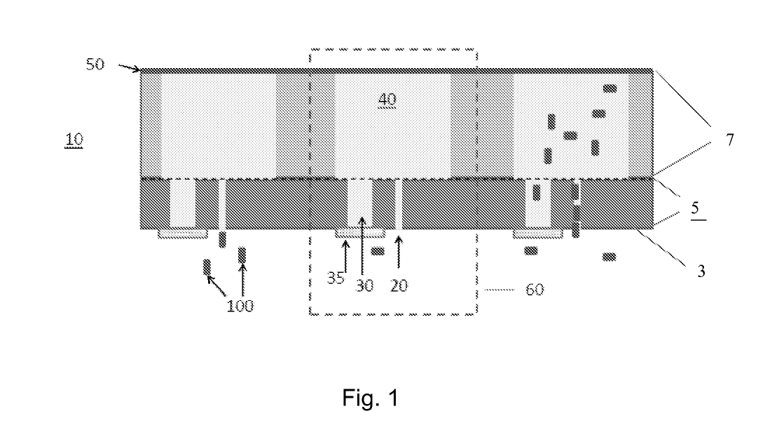

An embodiment of a device 10 for harvesting monocultures of microbial cells from an environment is depicted in FIG. 1. The device includes substrate 5 bonded to a lower side of upper body 7. The device contains a plurality of microbial isolation units 60 (three are depicted in FIG. 1). Each microbial isolation unit contains food chamber 40, nanochannel 20 (with opening at exposed surface 3), and aperture 30, which is sealed with nanoporous membrane 35. Bacteria 100 in the environment below the device enter the nanochannel and proliferate into the food chamber, forming a monoculture in the food chamber. The top of the device is sealed with lid 50 after filling of the chambers with a culture medium. The lid covers the food chambers and prevents entry of microbes into the food chamber from above.

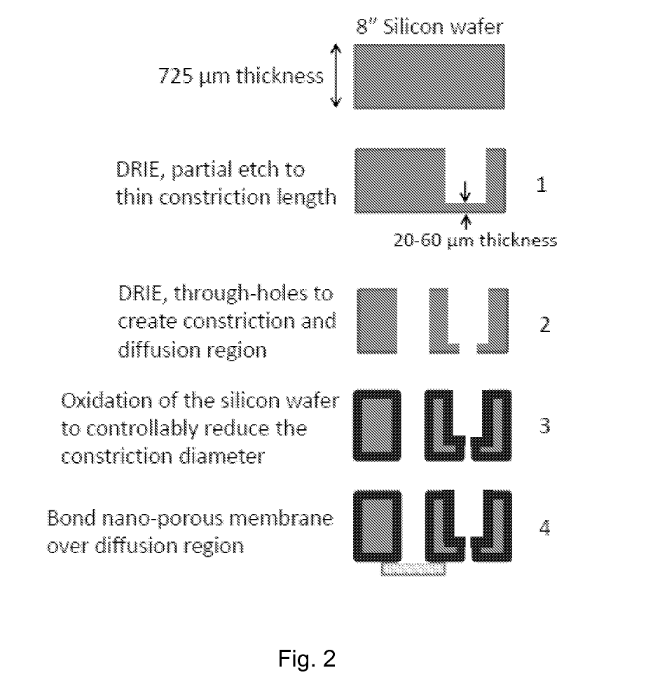

FIG. 2 depicts a process for fabrication of the substrate of an embodiment of the device. The figure depicts only a single nanopore and aperture; however, an desired number of nanopores and apertures, as well as additional optional nanochannels and/or microchannels, could be fabricated in parallel. First, a silicon wafer is thinned by deep reactive-ion etching (DRIE) in the areas intended for nanochannel production, so as to reduce the thickness of the wafer to correspond to the length of the nanochannel (e,g., 20-60 μm as shown in the figure). DRIE is a process capable of creating deep holes of high aspect ratio in silicon wafers and other materials. There are two major types of DRIE, one that uses a cryogenic process (see, e.g., en.wikipedia.org/wiki/Deep_reactive-ion_etching) and another that uses the Bosch process (see, e.g., U.S. Pat. Nos. 5,501,893; 6,531,068; and 6,284,148; all incorporated by reference herein). Next, a second, larger channel is cut through the wafer using DRIE to form the environmental exchange aperture, and the nanochannel is cut through the thinned region. In order to adjust the diameter (i.e., constriction) of the nanochannel to a value that is suitable for trapping microbial cells but allowing them to proliferate through the channel, the substrate is coated with an oxide layer (e.g., silicon dioxide, added by a chemical or physical deposition method) of sufficient thickness so as to form the desired nanochannel width. Finally, a nanoporous membrane is bonded over the aperture to complete the substrate structure.

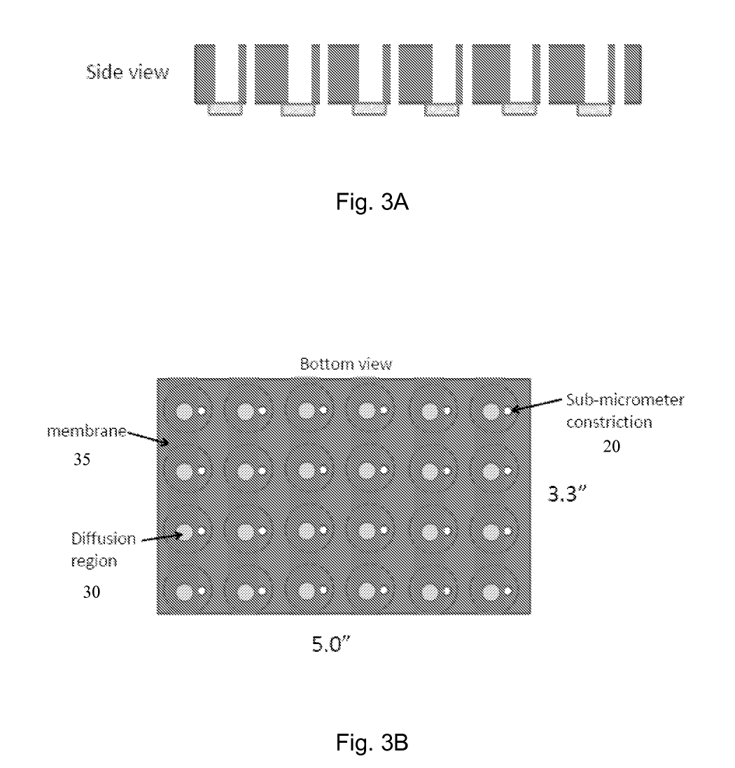

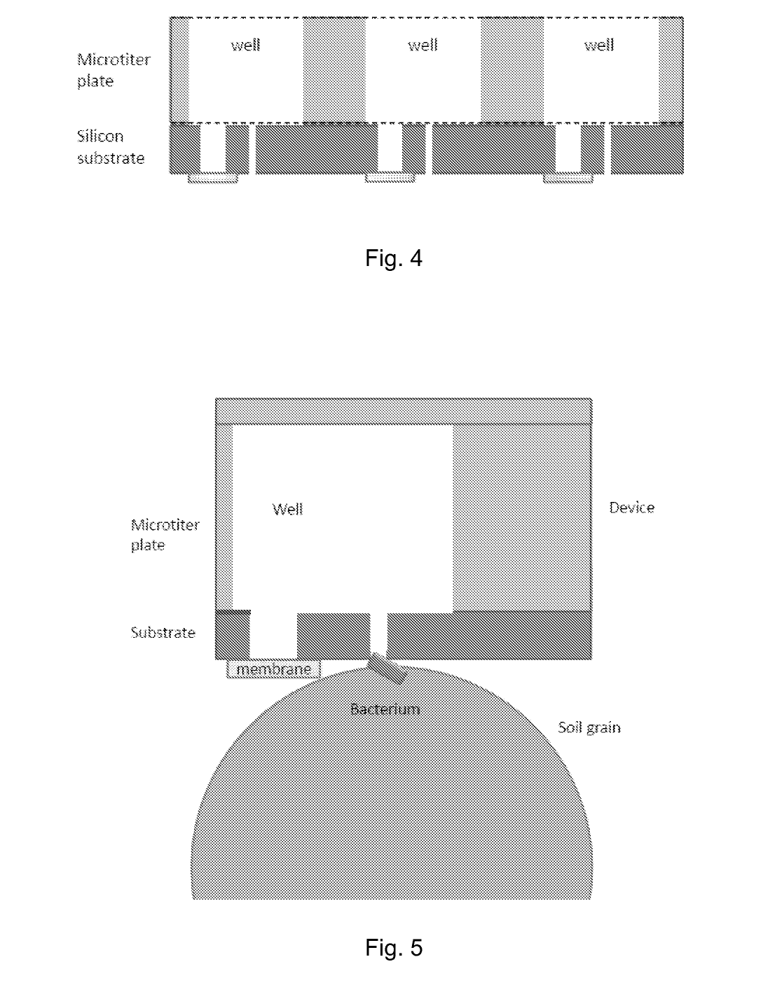

FIGS. 3A and 3B depict an embodiment of a completed substrate in side view (FIG. 3A) and in bottom view (FIG. 3B). This embodiment corresponds to a 24-well microtiter plate format. The substrate is then bonded to an upper body which is a single piece of molded plastic, such as polystyrene, in the form of a microtiter plate but lacking the bottom portions of the wells. The resulting device is depicted in FIG. 4. An upper body can be fabricated from a standard microtiter plate by cutting off the bottom portion, or by drilling holes in the well bottoms, or can be fabricated as an intact structure, for example, by injection molding. The bonding process can be performed, for example, using an adhesive, a double-sided tape, or a thermal annealing process, optionally involving plasma treatment of a plastic upper body to be bonded to a silicon substrate.

Due to the exposure of a nanochannel opening at a surface, the devices of the present invention are particularly suited for sampling of bacteria and other microbial cells from a solid material or a suspension of solid particles in a liquid or gas. As an example, FIG. 5 depicts a soil grain (e.g., 50 μm in diameter) in contact with a sampling surface of such a device, where a bacterium adsorbed on the soil grain is entering the nanochannel, from which its progeny will eventually form a monoculture in the food chamber (well). The structures shown in FIG. 5 are not to scale.

The invention provides a variety of methods of isolating and characterizing previously unknown or uncharacterized microbes such as bacteria from natural environments, as well as analyzing their products and their biochemistry. One such method is a method of isolating and culturing a single microbial cell from an environment to obtain a monoculture of the microbial cell. A device such as described above is placed into an environment suspected of containing microbial cells of interest. The device is placed into a suitable orientation, such that the sampling surface of the device, containing an open nanochannel, is exposed to a material of interest. A series of devices also can be placed simultaneously into similar or different environments in a given area. The device is maintained in the environment for a period of time expected to suffice for microbes to enter the nanochannels of the device, for its progeny to divide and eventually reach the food chamber, where they proliferate and form a monoculture. The device can then be removed from the environment, and the cultured microbes can be accessed using robotic fluid handling equipment, with which they are sub-cultured and analyzed. For example, the cells of a given monoculture can be plated onto a variety of growth media to ascertain their optimal growth conditions. DNA, proteins, or other biomolecules can be extracted from the monoculture and studied (e.g., sequenced, or analyzed by PCR, Western blotting or ELISA) to identify the species or strain of bacteria or other microbes present in the environment sampled. Small molecules such as potential antibiotics can be identified (e.g., by mass spectrometry) in the culture medium of the monocultures. Characterization of the metabolism or nutritional requirements of the microbial cells can lead to new industrial or pharmaceutical products or biochemical processes. Identification of microbial species and their natural reservoirs can lead to new insights in the spread of disease or microbial evolution.

Of particular interest is the use of the devices of the invention to identify new species of bacteria in environmental samples. These include Archebacteria that may live in extreme environments, Actinomycetes that could be a source of new antibiotics, and useful soil bacteria such as nitrogen-fixing bacteria, nitrifying bacteria, and denitrifying bacteria. The ability to use microtiter plate formats with hundreds or even thousands of different growth chambers on a single device enables a large variety of different growth media to be tested simultaneously, which can greatly simplify the work needed to find appropriate culture conditions for previously unknown species. Further, the use of a nanoporous environmental exchange membrane allows for the growth of microbial cells that depend critically on unknown chemical substances in their environment, and can lead to the identification of those substances by adding back various extracts and purified chemical substances into the environment, which then diffuse through the nanoporous membrane and alter the growth or survival of the microbes in the growth chamber. This can also be done using a variation of the devices described above, in which the nanochannel open to the environment is omitted; in that way, no new microbial cells can enter the growth chamber, which can be seeded with desired microbes at the outset. New antibiotics can be identified in this manner by observing a decline in the viability of cultured cells.

The methods of the invention also can be used to identify and culture new microbial species such as bacteria that degrade biomaterials in the environment, or in industrial or agricultural waste. This can lead to new monocultures or communities of microorganisms that more efficiently break down such waste, or that convert it into useful products. Studies of fecal matter using a device of the invention also can lead to useful medical information about individual nutrition or health, or can lead to the development of new or improved probiotic formulations.

Another method of the invention is a method of screening for previously unknown antibiotics produced by microorganisms in an environment. In this method, one or more food chambers of the device are pre-loaded with a pathogenic or disease causing microorganism in a growth medium. The device is then placed in contact with an environment suspected of harboring antibiotic-producing microbes. Later, the device is analyzed, and microbial cells that enter growth chambers containing the pathogenic microorganism and overgrow those chambers are identified as possibly producing an antibiotic substance useful in combatting the pathogenic microorganism.

This application claims the priority of U.S. Provisional Application No. 62/065,944 filed 20 Oct. 2014 and entitled “High Throughput Bacterial Isolation Using Sub-Micrometer Constrictions”, the whole of which is hereby incorporated by reference.

As used herein, “consisting essentially of ” does not exclude materials or steps that do not materially affect the basic and novel characteristics of the item. Any recitation herein of the term “comprising”, particularly in a description of components of a composition or in a description of elements of a device, can be exchanged with “consisting essentially of or “consisting of”.

While the present invention has been described in conjunction with certain preferred embodiments, one of ordinary skill, after reading the foregoing specification, will be able to effect various changes, substitutions of equivalents, and other alterations to the compositions and methods set forth herein.

1. A device for isolating and culturing single cells of a population of microbial cells from an environment, the device comprising:
a nanochannel comprising a first end disposed at a surface of the device exposed to an environment, the environment comprising a mixture of microbial cells; and
a food chamber fluidically coupled to a second end of the nanochannel; wherein the nanochannel has a cross-sectional diameter that allows entry of only a single microbial cell from the mixture of microbial cells and prevents the microbial cell from entering the food chamber, but allows only progeny of the single microbial cell to enter the food chamber.

2. The device of claim 1, wherein the nanochannel has a cross-sectional diameter in the range from about 250 nm to about 1000 nm and a length of from about 10 μm to about 80 μm.

3. The device of claim 1, wherein the nanochannel has a cross-sectional diameter of about 700 nm.

4. The device of claim 1, wherein the device is configured such that said surface is capable of contacting a solid material in said environment.

5. The device of claim 4, wherein the first end of the nanochannel is capable of contacting a solid material in said environment.

6. The device of claim 4 or claim 5, wherein the solid material is selected from the group consisting of soil, sand, biomass, sewage, sediment from a body of water, and rock.

7. The device of claim 6, wherein the solid material comprises particles having a diameter of about 50 μm or greater.

8. The device of claim 6, wherein the solid material is a particulate solid material suspended in water or air.

9. The device of claim 1, wherein the food chamber comprises an aperture covered by a nanoporous membrane at said surface, the nanoporous membrane exposed to said environment in use and allowing passage of nutrients from the environment but not allowing passage of microbial cells.

10. The device of claim 9, wherein the aperture has a diameter in the range from about 50 μm to about 500 μm.

11. The device of claim 9, wherein the nanoporous membrane comprises pores having a diameter from about 5 nm to about 100 nm.

12. The device of claim 11, wherein the nanoporous membrane is a polycarbonate or aluminum oxide membrane having pores of about 30 nm average diameter.

13. The device of claim 1 comprising a plurality of nanochannels and a plurality of food chambers, wherein each nanochannel comprises a first end disposed at said surface and a second end fluidically coupled to a unique one of said food chambers, each coupled nanochannel and food chamber defining a microbial isolation unit.

14. The device of claim 13, wherein the microbial isolation units are configured as a two-dimensional array.

15. The device of claim 14, wherein the array is in a microtiter plate format.

16. The device of claim 15, wherein the microtiter plate format is compatible with a robotic fluid handling device.

17. The device of claim 15, wherein the array comprises 24, 96, 384, or 1536 microbial isolation units.

18. The device of claim 15, wherein the food chambers are formed from the wells of a microtiter plate.

19. The device of claim 18, wherein well bottoms of said microtiter plate are formed by a substrate attached to a lower surface of the microtiter plate, the substrate comprising the nanochannels.

20. The device of claim 19, wherein the substrate comprises silicon, glass, or quartz.

21. The device of claim 14, wherein each food chamber comprises an aperture covered by a nanoporous membrane at said surface, the nanoporous membrane exposed to said environment in use and allowing passage of nutrients from the environment but not allowing passage of microbial cells.

22. The device of claim 21, wherein the apertures have a diameter in the range from about 50 μm to about 500 μm.

23. The device of claim 1, wherein the food chamber comprises a culture medium that supports the growth of at least one microbial cell of the population of microbial cells.

24. The device of claim 14, wherein the plurality of food chambers comprise one or more culture media.

25. The device of claim 1, wherein the microfluidic food chamber is fluidically coupled with one or more nanofluidic and/or microfluidic channels that permit exchange of a fluid medium within the food chamber and/or harvesting of microbial cells from the food chamber.

26. The device of claim 1, wherein the nanochannel and food chamber are empty spaces in a solid structure comprising a polymer material.

27. The device of claim 26, wherein the polymer material is polydimethylsiloxane (PDMS).

28. The device of claim 1, wherein the food chamber is an empty space in a polymer material, the nanochannel is an empty space in a silicon, glass, or quartz substrate, and the substrate is adhered to the polymer material such that the substrate forms a floor of the food chamber.

29. The device of claim 1, wherein the substrate further comprises an aperture covered by a nanoporous membrane, the nanoporous membrane exposed to said environment in use and allowing passage of nutrients from the environment but not allowing passage of microbial cells.

30. The device of claim 1, further comprising one or more valves, ports, holes, fluid reservoirs, pumps, vacuum lines, additional membranes, additional microfluidic channels, and/or additional nanochannels.

31. The device of claim 23 that is sealed from the environment but for said nanochannel.

32. The device of claim 24 that is sealed from the environment but for the plurality of nanochannels.

33. The device of claim 31 or claim 32 that is sterile and devoid of any viable microbial cells prior to placement in said environment.

34. A method of fabricating the device of claim 21, the method comprising the steps of:
(a) fabricating a substantially planar substrate comprising a nanochannel and a nanoporous aperture by the steps of:
(i) providing a substantially planar silicon, glass, or quartz substrate;
(ii) performing a first deep reactive ion etching from an upper side of the substrate to remove a plurality of first columns of material from said substrate, leaving a floor at a base of said columns, the floor having a thickness from about 20 to about 60 pm;
(iii) performing a second deep reactive ion etching to remove a plurality of second columns of material from said substrate, each second column adjacent to one of said first columns, the second columns extending the entire thickness of the substrate, and to perforate the floor of the first columns;
(iv) coating the substrate with an oxide layer, whereby the floor perforation of the first columns achieves a desired first diameter and the floor achieves a desired thickness, defining a single nanochannel in the floor of each of the plurality of first columns, each nanochannel having said first diameter and a length equal to the floor thickness, and whereby the second columns each create a plurality of apertures of a second diameter, each aperture adjacent to one of said nanochannels; and
(v) bonding a nanoporous membrane across each aperture at a lower surface of the substrate to form said nanoporous apertures;
wherein the nanochannels and apertures form a two dimensional array corresponding to a two dimensional array of wells in a microtiter plate; and
(b) bonding the substrate from (a) to a bottom side of a microtiter plate whose wells lack floors, whereby the substrate forms floors of wells of the microtiter plate to form said device; wherein the substrate is aligned with the wells such that a single nanochannel and a single aperture are present in the floor of each well.

35. The method of claim 34, further comprising:
(c) filling the wells with one or more culture media; and
(d) sealing the wells to form the device of claim 29.

36. The method of claim 34, wherein the bonding in step (b) comprises using an adhesive.

37. The method of claim 34, wherein the bonding in step (b) comprises plasma treatment of the microtiter plate.

38. The method of claim 34, wherein the bonding a nanoporous membrane across each aperture at a lower surface of the substrate in step (a)(v) comprises bonding a continuous strip of nanoporous membrane material across a plurality of said apertures arranged in a linear array.

39. The method of claim 34, wherein the microtiter plate is a one-piece molded plastic article in the form of a microtiter plate but lacking well bottoms.

40. The method of claim 34, wherein the microtiter plate has a format that is compatible with a robotic fluid handling device.

41. The method of claim 34, wherein the microtiter plate comprises 24, 96, 384, or 1536 wells.

42. The method of claim 35, wherein the wells are sealed with an optically transparent material.

43. The method of claim 34, further comprising installing in the device one or more valves, ports, holes, fluid reservoirs, pumps, vacuum lines, additional membranes, additional microfluidic channels, and/or additional nanochannels.

44. A method of isolating and culturing a single microbial cell to obtain a monoculture of microbial cells, the method comprising the steps of:
(a) depositing the device of claim 31, 32, or 33 into an environment comprising a mixture of microbial cells such that the surface of the device comprising the first end of said nanochannel contacts material of said environment suspected of comprising said microbial cells;
(b) allowing one of said mixture of microbial cells to migrate into the nanochannel of the device;
(c) maintaining the device under conditions suitable for allowing said microbial cell to divide within the nanochannel and produce progeny, whereby the progeny eventually enter the food chamber; and
(d) maintaining the device under conditions suitable for the progeny entering the food chamber to multiply in the food chamber, forming a monoculture of microbial cells.

45. The method of claim 44, further comprising:
(e) removing the device from said environment for analysis or sub-culturing of the microbial cells.

46. The method of claim 45, wherein the analysis comprises DNA sequence analysis of the microbial cells.

47. The method of claim 45, wherein the analysis comprises characterizing the metabolism or nutritional requirements of the microbial cells.

48. The method of claim 45, wherein the device is maintained in the environment for a period of days, weeks, or months before removal from the environment.

49. The method of claim 44, wherein the microbial cells are bacteria.

50. The method of claim 49, wherein the bacteria are anaerobic bacteria.

51. The method of claim 49, wherein the bacteria are Actinomycetes, Archebacteria, nitrogen-fixing bacteria, nitrifying bacteria, denitrifying bacteria

52. The method of claim 44, wherein the device comprises a plurality of microfluidic food chambers, each fluidically coupled to a single channel opening on said surface.

53. The method of claim 52, wherein a plurality of monocultures are obtained, each grown in a distinct food chamber and derived from a distinct single microbial cell of the environment.

54. The method of claim 53, whereby monocultures of two or more different species of microbes are obtained.

55. The method of claim 44, further comprising the step of:
(e) harvesting cells of the monoculture from the food chamber.

56. The method of claim 44, wherein the nanoporous membrane permits entry of nutrients from said environment.

57. The method of claim 56, wherein steps (c) and/or (d) are performed while supplying one or more additional nutrients through the membrane.

58. The method of claim 57, wherein the microbial cells only grow and form a monoculture when the material obtained from the natural environment is placed in contact with the membrane.

59. The method of claim 52, wherein the device comprises a plurality of food chambers, each food chamber containing a different culture medium, and whereby microbial cells from the mixture are identified based on their ability to grow in one or more of the food chambers.

60. The method of claim 44, wherein any step of the method is monitored using light microscopy to observe the presence or identity of microbial cells in the nanochannel or the food chamber.

61. A method of characterizing an effect of a chemical agent on the growth and/or survival of a population of microbial cells, the method comprising the steps of:
(a) forming a monoculture of microbial cells using the method of claim 44;
(b) supplying a chemical agent to the environment in which the device is deposited and allowing the agent to diffuse through the nanoporous membrane into the food chamber; and
(c) characterizing an effect of the chemical agent on the physiology and/or growth of the microbial cells in the food chamber.

62. The method of claim 61, wherein the chemical agent is an antibiotic or is suspected of having antibiotic activity.

63. A method of characterizing an effect of a chemical agent on the growth and/or survival of a population of microbial cells, the method comprising the steps of:
(a) forming a monoculture of microbial cells using the method of claim 56;
(b) sub-culturing the microbial cells from (a) into a device comprising a growth chamber, the growth chamber comprising an aperture covered by a nanoporous membrane;
(c) depositing the device containing the sub-culture into an environment comprising or suspected of comprising a chemical agent diffusible through the nanoporous membrane; and
(d) characterizing an effect of the chemical agent on the physiology and/or growth of the microbial cells in the growth chamber.

64. A method of isolating and/or identifying a microbial species or strain that metabolizes a chemical agent or degrades a biomaterial, the method comprising the steps of:
(a) performing the method of claim 44, wherein one or more food chambers of the device are preloaded with the chemical agent or the biomaterial;
(b) removing the device from the environment; and
(c) analyzing, isolating, or sub-culturing microbial cells whose survival and/or growth was enhanced in the presence of the chemical agent or the biomaterial in the device.

65. A method to aid in the identification of antibiotic-producing microbial cells, the method comprising the steps of:
(a) performing the method of claim 44, wherein one or more food chambers of the device are preloaded with a target pathogenic microbe;
(b) removing the device from the environment; and
(c) analyzing, isolating, or sub-culturing microbial cells that overgrow the pathogenic microbe in the device.
